SEQUENCE LISTING (13 sequences)

SEQ ID NO: 1 - protein, 5 aa, synthetic construct
SYAMS

SEQ ID NO: 2 - protein, 16 aa, synthetic construct
SIGSGGGTYYPDSVKD

SEQ ID NO: 3 - protein, 13 aa, synthetic construct
AYYRNHRGSVMDY

SEQ ID NO: 4 - protein, 15 aa, synthetic construct
KASQTVDYDGDSYMN

SEQ ID NO: 5 - protein, 7 aa, synthetic construct
SASNLES

SEQ ID NO: 6 - protein, 9 aa, synthetic construct
QQSTEDPPT

SEQ ID NO: 7 - protein, 231 aa, synthetic construct
PGLVKPSQSLSLTCSVTGYSITTNYNWNWIRQFPGNKLEWMGYIRYDGTSEYTPSLKNRVSITRDTSMNQFFLRLTSVTPEDTATYYCARLDYWGQGTLVTVSSGGGGSGGGGSGGGGSDIVMTQNPLSLPVSLGDEASISCRSSQSLENSNGNTFLNWFFQKPGQSPQLLIYRVSNRFSGVPDRFSGSGSGTDFTLKISRVEAEDLGVYFCLQVTHVPYTFGGGTTLEIK

SEQ ID NO: 8 - protein, 19 aa, synthetic construct
PSTKGPSVFPLAPSSSGSG

SEQ ID NO: 9 - protein, 28 aa, synthetic construct
METDTLLLWVLLLWVPGSTGDIQLQQSG

SEQ ID NO: 10 - protein, 132 aa, Homo sapiens
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSED

SEQ ID NO: 11 - protein, 410 aa, synthetic construct
METDTLLLWVLLLWVPGSTGDIQLQQSGPGLVKPSQSLSLTCSVTGYSITTNYNWNWIRQFPGNKLEWMGYIRYDGTSEYTPSLKNRVSITRDTSMNQFFLRLTSVTPEDTATYYCARLDYWGQGTLVTVSSGGGGSGGGGSGGGGSDIVMTQNPLSLPVSLGDEASISCRSSQSLENSNGNTFLNWFFQKPGQSPQLLIYRVSNRFSGVPDRFSGSGSGTDFTLKISRVEAEDLGVYFCLQVTHVPYTFGGGTTLEIKPSTKGPSVFPLAPSSSGSGQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSED

SEQ ID NO: 12 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 13 - DNA, 1236 bp, synthetic construct
  source 1..1236: mol_type = other DNA
atggagacagacacactcctgctatgggtgctgctgctctgggttccaggttccaccggtgatatacagcttcagcagtcaggacctggcctcgtgaaaccttctcagtctctgtctctcacctgctctgtcactggctactccatcaccactaattataactggaactggatccggcagtttccaggaaacaaactggaatggatgggctacataaggtacgacggtactagcgagtacaccccatctctcaaaaatcgagtctccatcactcgtgacacatctatgaatcagtttttcctgagattgacttctgtgactcctgaggacacagcgacgtattattgtgcaaggttggactactggggccaagggactctggtcaccgtctcctcaggtggaggcggttcaggcggaggtggctctggcggtggcggatcggacatcgttatgacgcagaatccactctccctgcctgtcagtcttggagatgaagcctccatctcttgcaggtcaagtcagagtcttgaaaacagtaatggaaacacctttttgaactggttcttccagaaaccaggccagtctccacagctcctgatctacagggtttccaaccgattttctggggtcccagacaggttcagcggtagtggatcagggacagatttcacactgaagatcagcagagtggaggctgaggatttgggagtttatttctgcctccaagttacacatgtcccgtacacgttcggaggggggaccacgctggaaataaaaccctccaccaagggcccatctgtcttccccctggcccccagctcctctggctccggacaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctga

(19) United States
(12) Patent Application Publication
(10) Pub. No.: US 2025/0017965 A1
(43) Pub. Date: Jan. 16, 2025

(54) ANTI-CD40 SINGLE-CHAIN VARIABLE FRAGMENT AND HUMAN IL-21 FUSION PROTEIN (CD40SCFV-IL-21)

(71) Applicant: Wisconsin Alumni Research Foundation, Madison, WI (US)

(72) Inventors: Jacques GALIPEAU, Madison, WI (US); Andrea PENNATI, Madison, WI (US); Shala YUAN, Madison, WI (US)

(21) Appl. No.: 18/710,334

(22) PCT Filed: Nov. 15, 2022
(86) PCT No.: PCT/US2022/079890

§ 371 Date: May 15, 2024

Related U.S. Application Data
(60) Provisional application No. 63/279,425, filed on Nov. 15, 2021.

(51) Int. Cl. A61K 35/17; A61K 38/00; A61K 39/00; A61P 37/04; C07K 14/54; C07K 16/28; C12N 5/0781

(52) U.S. Cl. CPC A61K 35/17; A61K 39/4612; A61P 37/04; C07K 14/54; C07K 16/2878; C12N 5/0635; A61K 38/00; A61K 2239/38; C07K 2317/622; C07K 2319/00

(57) ABSTRACT

The present invention provides fusion polypeptides comprising an anti-CD40 scFv and IL-21. Also provided are methods for making and using the fusion polypeptides for the generation of B regulatory cells (B_{regs}), and methods of treatment using said B_{regs}.

### CROSS-REFERENCE TO RELATED PATENT APPLICATIONS

The present application claims priority to U.S. Provisional Patent Application No. 63/279,425 that was filed Nov. 15, 2021, the entire contents of which are hereby incorporated by reference.

### STATEMENT REGARDING FEDERALLY SPONSORED RESEARCH

Not applicable.

### SEQUENCE LISTING

A Sequence Listing accompanies this application and is submitted as an xml file of the sequence listing named “960296_04336.xml” which is 14,586 bytes in size and was created on Nov. 14, 2022. The sequence listing is electronically submitted via Patent Center and is incorporated herein by reference in its entirety.

### BACKGROUND

Regulatory B cells (B_{regs}) are important immunosuppressive cells that support immunological tolerance through the production of polarizing cytokines, including the expression of IL-10, IL-35 and TGF-β. B_{regs} can suppress immunopathology by inhibiting the expansion of pathogenic T cells and other pro-inflammatory cells. The importance of human B_{regs} in the maintenance of immune homeostasis has been observed in a variety of immune-related pathologies, such as autoimmune diseases, cancers, and chronic infections that showed abnormalities in B_{regs} numbers and/or functions. Since B_{regs} make up only 1-2% of total cells in the blood, isolating and expanding their numbers naturally to develop therapeutic cells isn't feasible.

### SUMMARY

Provided herein are fusion polypeptides comprising an anti-CD40 scFv and IL-21 and methods for making and using the fusion polypeptides for the generation of B regulatory cells (B_{regs}), and methods of treatment using said B_{regs}.

In an aspect of the current disclosure, polypeptides are provided. In some embodiments, the polypeptides comprise from N to C terminus: an scFv specific for CD40 linked to IL-21. In some embodiments, the scFv comprises SEQ ID NO: 1-6 or has 95% identity to SEQ ID NO: 1-6. In some embodiments, the scFv comprises SEQ ID NO: 7 or has 90% identity to SEQ ID NO: 7. In some embodiments, the scFv is linked via a peptide bond to IL-21. In some embodiments, the scFv is linked to IL-21 via a linker peptide. In some embodiments, the linker comprises SEQ ID NO: 8 or has 90% identity with SEQ ID NO: 8. In some embodiments, the polypeptides further comprise a signal peptide. In some embodiments, the signal peptide comprises SEQ ID NO: 9 or has 90% identity with SEQ ID NO: 9. In some embodiments, IL-21 comprises SEQ ID NO: 10 or has 90% identity with SEQ ID NO: 10. In some embodiments, the polypeptide comprises SEQ ID NO: 11 or has 90% identity with SEQ ID NO: 11.

In another aspect of the current disclosure, pharmaceutical compositions are provided. In some embodiments, the pharmaceutical compositions comprise, from N to C terminus: an scFv specific for CD40 linked to IL-21 and a pharmaceutically acceptable carrier. In some embodiments, the scFv comprises SEQ ID NO: 1-6 or has 95% identity to SEQ ID NO: 1-6. In some embodiments, the scFv comprises SEQ ID NO: 7 or has 90% identity to SEQ ID NO: 7. In some embodiments, the scFv is linked via a peptide bond to IL-21. In some embodiments, the scFv is linked to IL-21 via a linker peptide. In some embodiments, the linker comprises SEQ ID NO: 8 or has 90% identity with SEQ ID NO: 8. In some embodiments, the polypeptides further comprise a signal peptide. In some embodiments, the signal peptide comprises SEQ ID NO: 9 or has 90% identity with SEQ ID NO: 9. In some embodiments, IL-21 comprises SEQ ID NO: 10 or has 90% identity with SEQ ID NO: 10. In some embodiments, the polypeptide comprises SEQ ID NO: 11 or has 90% identity with SEQ ID NO: 11.

In another aspect of the current disclosure, methods of generating B regulatory cells (B_{reg}) cells are provided. In some embodiments, the methods comprise contacting B cells isolated from a biological sample from a subject with a polypeptide comprising, from N to C terminus: an scFv specific for CD40 linked to IL-21, or a pharmaceutical composition comprising a polypeptide comprising from N to C terminus: an scFv specific for CD40 linked to IL-21; and a pharmaceutically acceptable carrier; and a TLR9 agonist for at least about 2 days in vitro. In some embodiments, the TLR9 agonist is CpG. In some embodiments, the scFv comprises SEQ ID NO: 1-6 or has 95% identity to SEQ ID NO: 1-6. In some embodiments, the scFv comprises SEQ ID NO: 7 or has 90% identity to SEQ ID NO: 7. In some embodiments, the scFv is linked via a peptide bond to IL-21. In some embodiments, the scFv is linked to IL-21 via a linker peptide. In some embodiments, the linker comprises SEQ ID NO: 8 or has 90% identity with SEQ ID NO: 8. In some embodiments, the polypeptides further comprise a signal peptide. In some embodiments, the signal peptide comprises SEQ ID NO: 9 or has 90% identity with SEQ ID NO: 9. In some embodiments, IL-21 comprises SEQ ID NO: 10 or has 90% identity with SEQ ID NO: 10. In some embodiments, the polypeptide comprises SEQ ID NO: 11 or has 90% identity with SEQ ID NO: 11. In some embodiments, the cells are contacted for about 3-5 days. In some embodiments, the B cells are contacted with the polypeptide or the composition and the TLR agonist at substantially the same time. In some embodiments, the B cells are contacted with the TLR agonist before the polypeptide or the composition. In some embodiments, the biological sample is peripheral blood, plasma, or derived from leukapheresis. In some embodiments, the B cells are isolated from peripheral blood mononuclear cells (PBMCs) or from leukapheresis. In some embodiments, the B cells are isolated from a sample that has been cryopreserved. In some embodiments, the B cells are further cryopreserved after the contacting step.

In another aspect of the current disclosure, methods of treating a subject are provided. In some embodiments, the methods comprise administering cells generated by contacting B cells isolated from a biological sample from a subject with a polypeptide comprising, from N to C terminus: an scFv specific for CD40 linked to IL-21, or a pharmaceutical composition comprising a polypeptide comprising from N to C terminus: an scFv specific for CD40 linked to IL-21; and a pharmaceutically acceptable carrier; and a TLR9 agonist for at least about 2 days in vitro, to a subject in an amount sufficient to treat the subject, wherein the subject is in need of treatment with regulatory B cells. In some embodiments, the scFv comprises SEQ ID NO: 1-6 or has 95% identity to SEQ ID NO: 1-6. In some embodiments, the scFv comprises SEQ ID NO: 7 or has 90% identity to SEQ ID NO: 7. In some embodiments, the scFv is linked via a peptide bond to IL-21. In some embodiments, the scFv is linked to IL-21 via a linker peptide. In some embodiments, the linker comprises SEQ ID NO: 8 or has 90% identity with SEQ ID NO: 8. In some embodiments, the polypeptides further comprise a signal peptide. In some embodiments, the signal peptide comprises SEQ ID NO: 9 or has 90% identity with SEQ ID NO: 9. In some embodiments, IL-21 comprises SEQ ID NO: 10 or has 90% identity with SEQ ID NO: 10. In some embodiments, the polypeptide comprises SEQ ID NO: 11 or has 90% identity with SEQ ID NO: 11. In some embodiments, the cells are contacted for about 3-5 days. In some embodiments, the B cells are contacted with the polypeptide or the composition and the TLR agonist at substantially the same time. In some embodiments, the B cells are contacted with the TLR agonist before the polypeptide or the composition. In some embodiments, the biological sample is peripheral blood, plasma, or derived from leukapheresis. In some embodiments, the B cells are isolated from peripheral blood mononuclear cells (PBMCs) or from leukapheresis. In some embodiments, the B cells are isolated from a sample that has been cryopreserved. In some embodiments, the B cells are further cryopreserved after the contacting step. In some embodiments, the subject is in need of treatment for an inflammatory or autoimmune disease or disorder, or a tissue injury syndrome. In some embodiments, the inflammatory or autoimmune disease or disorder is selected from the group consisting of: multiple sclerosis (MS), inflammatory colitis, radiation injury, and amyotrophic lateral sclerosis (ALS). In some embodiments, the subject is in need of treatment for MS, and the method induces re-myelination of the peripheral nerves damaged by MS. In some embodiments, the number of cells administered to the subject is about 1×10⁶ to 1×10⁷ cells/kg.

In another aspect, methods of treating a subject by administering the fusion polypeptide or the pharmaceutical composition comprising the fusion polypeptide provided herein to a subject in an amount sufficient to treat the subject. The subject is in need of treatment with regulatory B cells to treat an inflammatory, autoimmune disease or disorder as detailed above.

In another aspect of the current disclosure, constructs are provided. In some embodiments, the constructs comprise a polynucleotide encoding a polypeptide comprising, from N to C terminus: an scFv specific for CD40 linked to IL-21. In some embodiments, the scFv comprises SEQ ID NO: 1-6 or has 95% identity to SEQ ID NO: 1-6. In some embodiments, the scFv comprises SEQ ID NO: 7 or has 90% identity to SEQ ID NO: 7. In some embodiments, the scFv is linked via a peptide bond to IL-21. In some embodiments, the scFv is linked to IL-21 via a linker peptide. In some embodiments, the linker comprises SEQ ID NO: 8 or has 90% identity with SEQ ID NO: 8. In some embodiments, the polypeptides further comprise a signal peptide. In some embodiments, the signal peptide comprises SEQ ID NO: 9 or has 90% identity with SEQ ID NO: 9. In some embodiments, IL-21 comprises SEQ ID NO: 10 or has 90% identity with SEQ ID NO: 10. In some embodiments, the polypeptide comprises SEQ ID NO: 11 or has 90% identity with SEQ ID NO: 11. In some embodiments, the constructs further comprise a promoter operably linked to the polynucleotide. In some embodiments, the polynucleotide comprises SEQ ID NO: 13.

In another aspect of the current disclosure, cells are provided. In some embodiments, the cells comprise a construct comprising a polynucleotide encoding a polypeptide comprising, from N to C terminus: an scFv specific for CD40 linked to IL-21 and are capable of producing the polypeptide. In some embodiments, the scFv comprises SEQ ID NO: 1-6 or has 95% identity to SEQ ID NO: 1-6. In some embodiments, the scFv comprises SEQ ID NO: 7 or has 90% identity to SEQ ID NO: 7. In some embodiments, the scFv is linked via a peptide bond to IL-21. In some embodiments, the scFv is linked to IL-21 via a linker peptide. In some embodiments, the linker comprises SEQ ID NO: 8 or has 90% identity with SEQ ID NO: 8. In some embodiments, the polypeptides further comprise a signal peptide. In some embodiments, the signal peptide comprises SEQ ID NO: 9 or has 90% identity with SEQ ID NO: 9. In some embodiments, IL-21 comprises SEQ ID NO: 10 or has 90% identity with SEQ ID NO: 10. In some embodiments, the polypeptide comprises SEQ ID NO: 11 or has 90% identity with SEQ ID NO: 11. In some embodiments, the constructs further comprise a promoter operably linked to the polynucleotide. In some embodiments, the polynucleotide comprises SEQ ID NO: 13.

In another aspect of the current disclosure, kits, systems, and platforms are provided. In some embodiments, the kits, systems, and platforms comprise a polypeptide comprising from N to C terminus: an scFv specific for CD40 linked to IL-21; and a TLR9 agonist. In some embodiments, the scFv comprises SEQ ID NO: 1-6 or has 95% identity to SEQ ID NO: 1-6. In some embodiments, the scFv comprises SEQ ID NO: 7 or has 90% identity to SEQ ID NO: 7. In some embodiments, the scFv is linked via a peptide bond to IL-21. In some embodiments, the scFv is linked to IL-21 via a linker peptide. In some embodiments, the linker comprises SEQ ID NO: 8 or has 90% identity with SEQ ID NO: 8. In some embodiments, the polypeptides further comprise a signal peptide. In some embodiments, the signal peptide comprises SEQ ID NO: 9 or has 90% identity with SEQ ID NO: 9. In some embodiments, IL-21 comprises SEQ ID NO: 10 or has 90% identity with SEQ ID NO: 10. In some embodiments, the polypeptide comprises SEQ ID NO: 11 or has 90% identity with SEQ ID NO: 11. In some embodiments, the TLR9 agonist comprises CpG.

### BRIEF DESCRIPTION OF THE DRAWINGS

FIG. 1 Plasmid map harboring the fusion transgene including the single chain variable fragment of anti-human CD40 and IL-21.

FIG. 2 Amino acid sequence of CD40ScFv-IL21 fusion protein (SEQ ID NO: 11), 441 amino acid, 45.37 KDa. Signal peptide (underlined), CD40ScFv (in normal font) and IL-21 (bold font); linker between the two protein is in italics.

FIGS. 3A and 3B Phenotype of B cells (dashed black), B cells treated with CpG alone (B-CpG, grey shade) and B_{regs} generated by contacting isolated human B cells with the CD40scFV-IL-21 fusion protein (solid black). (B): IL-10 expression detected by ELISA. ND: not detected, *<0.5.

FIG. 4 Expression of Granzyme B (GzmB) was detected by Western blotting.

FIG. 5 Phenotype of peripheral macrophages co-cultured with B_{regs} for 2 days detected by flow cytometry expression of CD16 and CD163. B cells (solid black), B_{regs} (dashed line) and unstimulated cells grey area.

FIGS. 6A and 6B (A) percentage of body weight loss post X-ray irradiation, and (B) overall survival curve after administration of B_{regs} or PBS alone.

FIG. 7 shows T cell suppression assay. (A) Human PBMCs were isolated from whole blood and stimulated with PHA. After 3 days the cells were collected and stained for Ki-67 and CD3 and gated based on isotype control on stimulated T cells. (B) Bar graphs of T cells proliferation suppression, statistical analysis has been carried out with 5 biological replicates. Data are expressed as the mean-SEM and analyzed by Student's t-test, *p<0.05, ** p<0.01, **** p<0.0001.

FIG. 8 shows the modulation of monocyte-derived macrophage phenotype. Expression of TNF-α, upon culture of CD14-LPS stimulated cells with control media (no sup), B cell supernatant (B sup), B-CpG supernatant (B-CpG sup) or B_{regs} supernatant (B_{regs} sup). Cytokine expression was measured by ELISA. Data are expressed as the mean-SEM and analyzed by Student's t-test, *** P<0.01, *** P<0.001, **** P<0.0001<0.05.

### DETAILED DESCRIPTION

The present invention provides polypeptides comprising fusion proteins that combine a CD40-specific antibody single chain variable fragment (scFV) fused to the cytokine interleukin 21 (IL-21). In addition, the present disclosure provides pharmaceutical compositions comprising the above CD40 scFv fused to IL-21 and a pharmaceutically acceptable carrier. Methods of using the above polypeptides and pharmaceutical compositions in the generation of human B_{regs} are also provided, along with methods of using the generated B_{regs} in treatment of disease.

Regulatory B cells (B_{regs}) are important immunosuppressive cells that support immunological tolerance through the production of polarizing cytokines, including the expression of IL-10, IL-35 and TGF-β (1-5). B_{regs} can suppress immunopathology by inhibiting the expansion of pathogenic T cells and other pro-inflammatory lymph-myeloid cells (6). The importance of human B_{regs} in the maintenance of immune homeostasis arises from a variety of immune-related pathologies, such as autoimmune diseases, cancers, and chronic infections that showed abnormalities in B_{regs} numbers and/or functions. The frequency of B_{regs} in blood is less than 1-2% (7) and numerical and functional B_{regs} defects have been described in several autoimmune diseases, including systemic lupus erythematosus (SLE), rheumatoid arthritis (RA), multiple sclerosis (MS), and psoriasis (8).

Though native endogenous B_{regs} are rare, in experimental mice it is technically feasible to harvest normal B-cells from blood and convert them in vitro to IL-10 competent B_{regs} in large number. These manufactured B_{regs} can be transfused as a cell pharmaceutical to affect outcomes of inflammatory disease. Indeed, the inventors have published data from mouse models of inflammatory disease (multiple sclerosis/EAE, inflammatory colitis, radiation injury and Amyotrophic Lateral Sclerosis (ALS)) (9-14) that transfusion of manufactured B_{regs} leads to profound improvement of clinical outcomes. However, a major obstacle in translational use of B_{regs} for humans as an investigational pharmaceutical is that conversion of human B-cells to B_{regs} is unresponsive to techniques developed for genesis of mouse B_{regs}.

The inventors disclose herein a novel polypeptide, CD40scFv-IL-21, that allows for efficient and robust conversion of human B-cells to IL-10 competent functional B_{regs} in vitro at suitable scale for investigational and pharmacological use. This novel synthetic polypeptide would allow for manufacture of personalized B_{regs} for use as a cellular pharmaceutical to treat inflammatory disorders with unmet medical needs, for example, multiple sclerosis. The inventors have also developed an in vitro potency assay predicated on the ability of B_{regs} to make IL-10 AND polarize macrophages to an M2 functionality in vitro. The inventors also have pilot data that human B_{regs} generated with the disclosed fusion protein can improve radiation injury outcomes in immune defective mice providing functional validation of the disclosed cell product generated in methods using the novel fusion protein.

Thus, herein the inventors demonstrate the possibility to convert B cells to IL-10 producing B cells with a regulatory phenotype by co-culturing B cells with a novel fusion protein, generated by coupling the single-chain variable fragment (scFv) of anti-human CD40 and human IL-21. The fusion transgene was synthetized and expressed in a lentiviral protein expression vector.

### Fusion Polypeptide:

In a first aspect, the present invention provides polypeptides. In some embodiments, the polypeptides comprise from N to C terminus: an scFv specific for CD40 linked to IL-21. The terms “scFv” or “single-chain variable fragment” are used interchangeably herein to refer to a polypeptide consisting of the fused variable regions of the heavy (V_{H}) and light (V_{L}) chains of immunoglobulins. In some embodiments, the V_{H} and V_{L} chains are fused to either side of a short peptide linker. In some embodiments, the scFv specific for CD40 comprises the CDR sequences of SEQ ID NOs: 1-6, or sequences 90%, 92%, 94%, 95%, 96%, 97%, 98%, 99% similar or identical to SEQ ID NOs: 1-6. In some embodiments, the scFv specific for CD40 has the sequence of SEQ ID NO: 7, or a sequence 90%, 92%, 94%, 95%, 96%, 97%, 98%, 99% similar or identical to SEQ ID NO: 7. CD40 is a tumor necrosis factor (TNF) receptor superfamily member expressed on antigen-presenting cells (APCs), including dendritic cells (DCs), B cells, macrophages, and monocytes and plays a key role in the activation of the immune system. CD40 is also expressed on a variety of non-hematopoietic cell types, including neuronal, epithelial, and endothelial cells and fibroblasts. Wild-type Human CD40 has the sequence of SEQ ID NO: 12. Other human CD40 agonistic antibodies are known in the art, including other scFv anti-CD40 polypeptides. Linscott's Directory of antibodies and Genbank have multiple antibodies that could be used by one of skill in the art in place of the particular sequence used here.

IL-21 is a cytokine produced by T cells and natural killer cells that has pleiotropic actions on a wide range of immune and non-immune cell types. In some embodiments, IL-21 has the sequence of SEQ ID NO: 10, or a sequence 90%, 92%, 94%, 95%, 96%, 97%, 98%, 99% similar or identical to SEQ ID NO: 10.

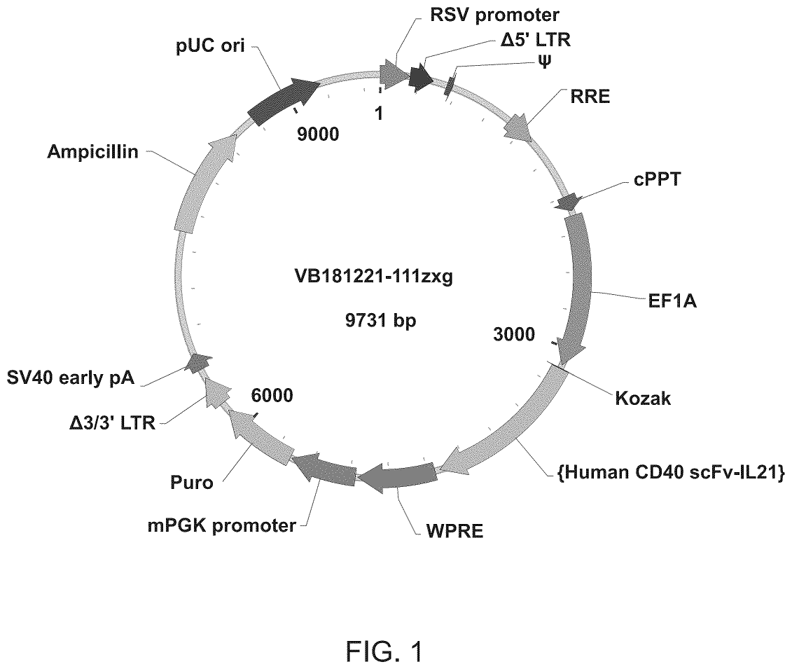

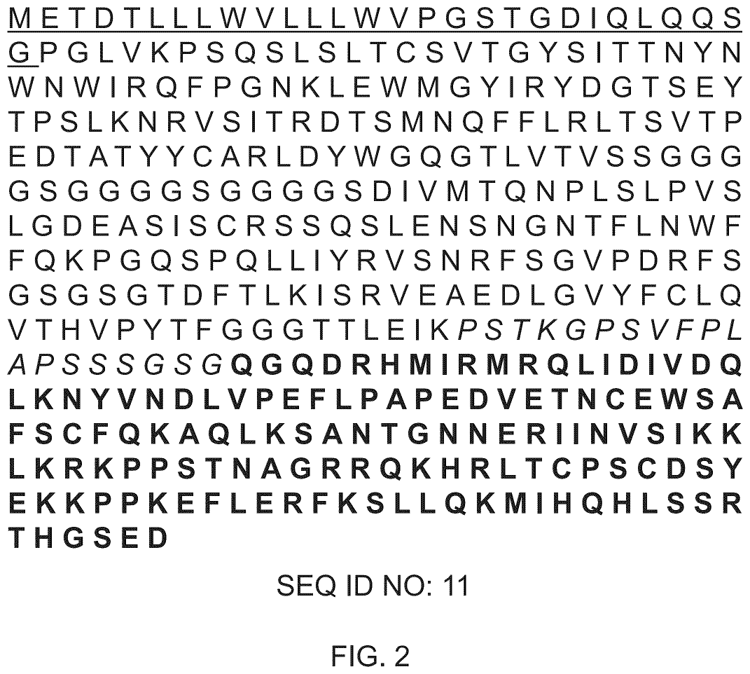

The polypeptide of the current invention comprises an scFv specific for CD40 linked to IL-21. As used herein, “linked” refers to the two peptides or polypeptides being covalently linked by amide peptide bond. In some embodiments, the CD40 is linked to IL-21 by a linker peptide, which has, for example, the sequence of SEQ ID NO: 8 or a sequence 90%, 92%, 94%, 95%, 96%, 97%, 98%, 99% similar or identical to SEQ ID NO: 8. In addition, the polypeptide of the instant disclosure is secreted by cells as a means to efficiently produce the fusion protein. Therefore, the polypeptide further comprises a signal sequence, which has, for example, the sequence of SEQ ID NO: 9, or a sequence 90%, 92%, 94%, 95%, 96%, 97%, 98%, 99% similar or identical to SEQ ID NO: 9. The sequence of the polypeptide may be, for example, SEQ ID NO: 11, or sequence 90%, 92%, 94%, 95%, 96%, 97%, 98%, 99% similar or identical to SEQ ID NO: 11 (FIGS. 1 and 2). The fusion proteins described herein may further include an epitope tag or a detectable label to aid in detection of or purification of the fusion protein. Epitope tags such as a FLAG tag, His tag or other tag are available to those of skill in the art. The tag or detectable protein may be attached to the fusion protein via a cleavable linker such as a furin 2A site. Detectable proteins include proteins such as luciferase, green fluorescent protein (GFP) or any other detectable label.

Thus, the novel fusion proteins, disclosed herein, act as both an agonist of CD40 and as an agonist to IL-21 receptor (IL-21R). The inventors have experimentally demonstrated that, for full biological activity of the fusion protein, the CD40 scFv is, preferably, located at the N-terminus of the fusion protein and IL-21 is located at the C-terminus. Moreover, the inventors designed additional fusion proteins with the natural ligand of CD40, CD40 ligand (CD40L), linked to IL-21 which was also ineffective at stimulating the generation of B_{regs}. Therefore, the disclosed polypeptides have the surprising effect of being able to generate B_{regs} from isolated peripheral B cells.

The terms “protein”, “polypeptide”, and “peptide” are used interchangeably herein to refer to a polymer of amino acids. A “protein” typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

Regarding the polypeptides disclosed herein, the phrases “% sequence identity,” “percent identity,” or “% identity” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Polynucleotides encoding any one of the polypeptides disclosed herein are provided. As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand). The polynucleotides may be cDNA.

Polynucleotides homologous to the polynucleotides described herein are also provided. Those of skill in the art understand the degeneracy of the genetic code and that a variety of polynucleotides can encode the same polypeptide. In some embodiments, the polynucleotides (i.e., polynucleotides encoding the fusion polypeptides) may be codon-optimized for expression in a particular cell including, without limitation, an insect cell, a plant cell, bacterial cell, or fungal cell that may be used for production of the fusion polypeptide. While particular polynucleotide sequences are disclosed herein any polynucleotide sequences may be used which encode a desired form of the polypeptides described herein. The polynucleotides provided herein are non-naturally occurring sequences. Use of codon optimized sequences may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

In another aspect of the present invention, constructs are provided. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

The constructs provided herein may be prepared by methods available to those of skill in the art. Notably each of the constructs claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The constructs provided herein may include a promoter operably linked to any one of the polynucleotides described herein. The promoter may be a heterologous promoter. As used herein, the terms “heterologous promoter,” “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the disclosed polynucleotides are operably connected to the promoter. As used herein, a polynucleotide is “operably connected” or “operably linked” when it is placed into a functional relationship with a second polynucleotide sequence. For instance, a promoter is operably linked to a polynucleotide if the promoter is connected to the polynucleotide such that it may affect transcription of the polynucleotides. In various embodiments, the polynucleotides may be operably linked to at least 1, at least 2, at least 3, at least 4, at least 5, or at least 10 promoters.

Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant, animal, bacterial, fungal, or synthetic promoter. In mammalian cells, typical promoters include, without limitation, promoters for Rous sarcoma virus (RSV), human immunodeficiency virus (HIV-1), cytomegalovirus (CMV), SV40 virus, and the like as well as the translational elongation factor EF-1α promoter or ubiquitin promoter. Those of skill in the art are familiar with a wide variety of additional promoters for use in various cell types.

Vectors including any of the constructs or polynucleotides described herein are provided. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. In the Examples a lentiviral vector was used to express the polypeptide. Vectors may carry genetic elements, such as those that confer resistance to certain drugs or chemicals.

Cells including any of the polynucleotides, constructs, or vectors described herein are provided. Suitable “cells” that may be used in accordance with the present invention include eukaryotic or prokaryotic cells. Suitable eukaryotic cells include, without limitation, plant cells, fungal cells, and animal cells. Those of skill in the art are familiar with cell types that may be used for expression of polypeptides.

### Pharmaceutical Compositions:

In a second aspect, the present invention provides pharmaceutical compositions comprising a polypeptide comprising from N to C terminus: an scFv specific for CD40 linked to IL-21 and a pharmaceutically acceptable carrier. The pharmaceutical compositions may further include a TLR agonist, suitably a TLR9 agonist such as CpG or, for example, cavrotolimod, CMP-001, CpG-28, EnanDIM, IMO-2055, IMO-2125/tilsotolimod, MGN1703/lefitolimod, NZ-TLR9, PF-3512676, SD-101, or S-540956. The disclosed polypeptides may be used in pharmaceutical composition to generate B_{regs}. For example, B cells may be isolated from a sample taken from a subject and cultured with the disclosed pharmaceutical compositions to generate B_{regs}. The B_{regs} may then, in some embodiments, be infused into the subject in an amount sufficient to treat the subject. In some embodiments, the scFv specific for CD40 comprises the sequences of SEQ ID NOs: 1-6, or sequences 90% or more identical to SEQ ID NOs: 1-6. The scFv specific for CD40 has, for example, the sequence of SEQ ID NO: 7, or a sequence 90% or more, e.g., 91% or more, 92% or more, 93% or more, 94% or more, 95% or more, 96% or more, 97% or more, 98% or more, 99% or more, identical to SEQ ID NO: 7. IL-21 may have the sequence of SEQ ID NO: 10, or a sequence 90% or more, e.g., 91% or more, 92% or more, 93% or more, 94% or more, 95% or more, 96% or more, 97% or more, 98% or more, 99% or more, identical to SEQ ID NO: 10. The disclosed polypeptides comprise an scFv specific for CD40 linked to IL-21. The CD40 may be linked to IL-21 by a linker peptide, which has, for example, the sequence of SEQ ID NO: 8 or a sequence 90% or more, e.g., 91% or more, 92% or more, 93% or more, 94% or more, 95% or more, 96% or more, 97% or more, 98% or more, 99% or more, identical to SEQ ID NO: 8. The polypeptide of the instant disclosure may be secreted by cells. Therefore, the polypeptide may further comprise a signal sequence, which may have the sequence of SEQ ID NO: 9, or a sequence 90% or more, e.g., 91% or more, 92% or more, 93% or more, 94% or more, 95% or more, 96% or more, 97% or more, 98% or more, 99% or more, identical to SEQ ID NO: 9, for example. The sequence of the polypeptide may be, for example, SEQ ID NO: 11, or sequence 90% or more, e.g., 91% or more, 92% or more, 93% or more, 94% or more, 95% or more, 96% or more, 97% or more, 98% or more, 99% or more, identical to SEQ ID NO: 11.

Pharmaceutically acceptable carriers are known in the art and include, but are not limited to, diluents (e.g., Tris-HCl, acetate, phosphate), preservatives (e.g., Thimerosal, benzyl alcohol, parabens), solubilizing agents (e.g., glycerol, polyethylene glycerol), emulsifiers, liposomes, and nanoparticles. Pharmaceutically acceptable carriers may be aqueous or non-aqueous solutions, suspensions, or emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, or suspensions, including saline and buffered media.

The pharmaceutical compositions of the present invention may further include additives such as albumin or gelatin to prevent absorption to surfaces, detergents (e.g., Tween 20, Tween 80, Pluronic F68, bile acid salts), anti-oxidants (e.g., ascorbic acid, sodium metabisulfite), bulking substances or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels, etc.) or onto liposomes, microemulsions, micelles, milamellar or multilamellar vesicles, erythrocyte ghosts, or spheroplasts. The compositions may also be formulated in lipophilic depots (e.g., fatty acids, waxes, oils) for controlled or sustained release.

### Methods of Generating B_{regs}:

In a third aspect, the present invention provides methods for generating B regulatory cells (B_{regs}). In some embodiments, the methods of generating B_{regs} comprise contacting B cells isolated from a biological sample from a subject with (1) a polypeptide comprising from N to C terminus: an scFv specific for CD40 linked to IL-21 or a pharmaceutical composition comprising a polypeptide from N to C terminus: an scFv specific for CD40 linked to IL-21 and a pharmaceutically acceptable carrier, and (2) a Toll-like receptor 9 (TLR9) agonist for at least about 2 days in vitro. Suitably the cells are contacted for about 3-5 days in vitro. The unique properties of the disclosed polypeptides allow for the efficient generation of human IL-10 secreting B_{reg} cells in vitro. TLR9 agonists are known in the art and may comprise, for example, CpG nucleotides. The TLR9 agonist may comprise, for example, cavrotolimod, CMP-001, CpG-28, EnanDIM, IMO-2055, IMO-2125/tilsotolimod, MGN1703/lefitolimod, NZ-TLR9, PF-3512676, SD-101, or S-540956. The inventors have demonstrated that generation of B_{regs} by contacting B cells with the disclosed polypeptides and pharmaceutical compositions thereof, requires additionally contacting B cells with TLR9 agonist. Further, cells may be pre-treated with TLR9 agonist before being contacted with the CD40scFv-IL-21 polypeptide.

B_{regs} may be generated from, for example, peripheral circulating B cells. The B cells may be harvested from whole peripheral blood or leukapheresis of a subject in need of treatment with the B_{regs} and separated to obtain B cells. The B cells can be further isolated using a negative selection method. Negative selection methods are known in the art, for example, magnetic columns or beads. In typical negative selection systems, a cell mixture is contacted with a plurality of antibodies specific for every cell type except the target cell type. Thus, contacting the cell mixture with the plurality of antibodies labels the cells other than the target cells. The non-target cells can then be removed using various methods by linking the antibodies to a solid support, e.g., columns, plates, or beads. Thus, what is left after removal of non-target cells is a solution of cells that is highly enriched, preferably greater than 90% or more, e.g., 91% or more, 92% or more, 93% or more, 94% or more, 95% or more, 96% or more, 97% or more, 98% or more, 99% or more enriched for the target cell population.

The methods of the current disclosure provide several benefits over other methods of generating human B_{regs} known in the art. For example, a sample comprising B cells may be isolated from a donor and “banked”, i.e., frozen for short-term storage, e.g., 1 day, 1 week, 1 month, or long-term storage, e.g., greater than 1 month, greater than 2 months, greater than 3 months, greater than 4 months, greater than 5 months, greater than 6 months, greater than 7 months, greater than 8 months, greater than 9 months, greater than 10 months, greater than 11 months, greater than 12 months, greater than 18 months, greater than 24 months, greater than 36 months, greater than 48 months, greater than 60 months, greater than 72 months, greater than 84 months, greater than 90 months, or more. Appropriate samples may be, for example, a leukapheresis sample from a patient. Then, the banked sample comprising B cells may be thawed and B cells isolated by any method known in the art, preferably by negative selection, and used in the disclosed methods of generating B_{regs}. Moreover, the generated B_{regs} may then be further banked, allowing for thawing of the B_{regs} at the most effective time for their use in downstream applications, e.g., treatment of a subject in need thereof, or in preclinical testing.

A further advantage of the disclosed methods of generating B_{regs} is that the method does not require the use of “feeder cells”. Feeder cells are a cell line that is cultured in conjunction, i.e., in the same vessel with, a target cell line, or cell mixture. Feeder cells provide, by way of example but not by way of limitation, growth factors, cytokines, or other molecules that support the growth, expansion, differentiation, etc. of the target cell population. A drawback of feeder cells is that the feeder cell line may introduce contaminants into the solution comprising the target cells, for example, foreign antigens, DNA, etc. that complicate or prevent their use in good manufacturing practice (GMP) production of drugs or, in this case, cellular therapies.

In a further embodiment, the B_{regs} may also be generated in vivo by administration of the polypeptide or pharmaceutical compositions comprising the fusion polypeptide directly to a subject or to a site within a subject.

Methods of Treatment Using B_{regs} Generated with the Disclosed Methods:

In a fourth aspect, the present invention provides methods for treating a subject. The methods comprise administering cells generated by the methods of the current disclosure to a subject in an amount sufficient to treat the subject, wherein the subject is in need of treatment with regulatory B cells (B_{regs}). As discussed above, peripheral B cells may be extracted from a subject using, for example, a leukapheresis procedure which typically results in about 1×10⁹ B cells. In some embodiments of the methods, the leukapheresis samples may be cryogenically banked before isolation of B cells. In other embodiments of the methods, the B cells are directly isolated from the sample without banking or freezing. Next, the isolated B cells may be contacted with the disclosed polypeptide (CD40scFv-IL-21) and a TLR9 agonist for about 2-6 days to generate B_{regs}. The prepared B_{regs} may be cryopreserved or used immediately after generation. As used herein, “cryopreserved” refers to storing cells, tissues, etc., below the freezing point of water, e.g., at below 0 deg. C., below −20 deg. C., or below −70 deg. C.

The B_{regs} generated using the disclosed methods and polypeptides have been demonstrated to have beneficial properties for the treatment of diseases or disorders resulting from an over-active immune response, for example, autoimmune diseases, tissue injury syndromes. A subject in need of treatment with B_{regs} may be a subject suffering from, for example, type I diabetes melitus, transplantation rejection, multiple sclerosis (MS), premature ovarian failure, scleroderma, Sjogren's disease, lupus, vitiligo, alopecia, polyglandular failure, Grave's disease, hypothyroidism, polymyosititis, pemphigus, Crohn's disease, colitis, autoimmune hepatitis, hypopituitarism, myocardititis, Addison's disease, autoimmune skin diseases, uveitis, pernicious anemia, hypoparathyroidism, rheumatoid arthritis, and other autoimmune diseases or disorders. A subject in need of treatment with B_{regs} may be suffering from, for example, multiple sclerosis (MS), inflammatory colitis, radiation injury, or amyotrophic lateral sclerosis (ALS). Without being limited by any theory or mechanism, it is believed that the B_{regs} generated by the disclosed methods produce cytokines, e.g., IL-10, and secrete other factors, e.g., granzymes, that down-regulate the host immune response. Effectively, the B_{regs} are able to “reset” the host immune system to a more favorable condition. For example, the inventors have demonstrated that B_{regs} can induce re-myelination of damaged nerves in a model of multiple sclerosis. Therefore, the B_{regs} generated by the disclosed methods offer significant advantages over current therapies for individuals in need of treatment thereof. A further advantage of the disclosed treatment method using B_{regs} is that the B_{regs} may be used as an “autologous” transfer, meaning the cells are of host-origin. Therefore, treatment of a subject with autologous B_{regs}, generated using the disclosed methods, will not produce any associated graft versus host effects or immune response thereto from the subject. An “immune response” is the reaction of the body to the presence of a foreign substance (i.e., an antigen). The treatment benefit conferred by the present methods may be evaluated by measuring a reduction in clinical signs, e.g., the mortality, morbidity, temperature, physical condition, or overall health of the subject.

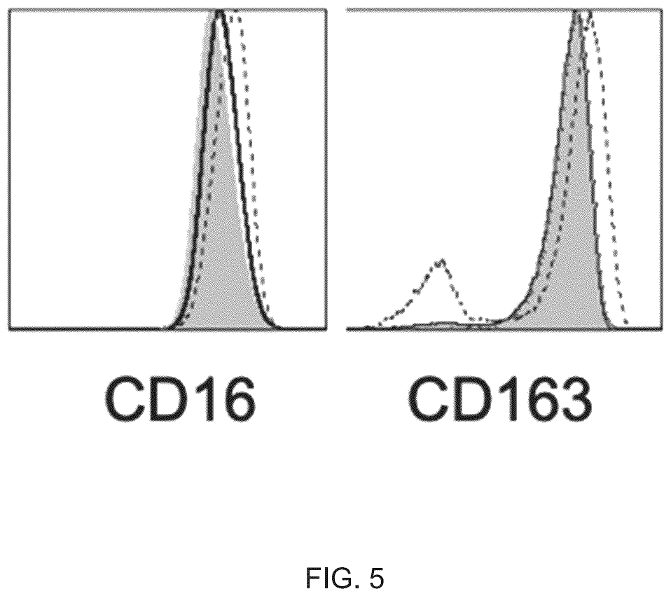

To evaluate the potentially efficacy of the disclosed cell therapies comprising B_{regs}, the inventors also disclose herein assays to evaluate the function of the cultured B_{regs}. Thus, because each lot of the cell product is a “bespoke” cell product, activity of the B_{reg} cell therapies can be evaluated in a quantitative manner. Briefly, co-culture of B_{regs} with human monocytes results in upregulation of CD16 and CD163 on the monocytes that is characteristic of exposure to functional, i.e., biologically active B_{regs} (FIG. 5). Therefore, B_{regs} may be evaluated based on their ability to polarize human monocytes in vitro, which can then inform the skilled artisan as to the potential efficacy or “activity” of the cell therapy. As used herein, activity refers to the level to which the cell therapy acts upon its intended target, e.g., the immune system.

As used herein, the terms “administering” and “administration” refer to any method of providing a pharmaceutical preparation to a subject. Suitable routes of administration include, without limitation, intravenous, and intrathecal routes. In some embodiments, the B_{regs} are administered intravenously. The B_{regs} can be administered as a single dose or in multiple doses. For example, the B_{regs} may be administered two or more times separated by about one day, two days, three days, four days, one week, two weeks, or by three or more weeks, a month, six months, or 12 months, or any period in between the indicated ranges as needed to treat the subject. While dosage of the cells provide herein may vary depending on the disease or condition being treated or the subject, in some embodiments the number of cells administered to the subject is about 1×10⁶ to 1×10⁷ cells/kg.

The “subject” to which the present methods are applied may be any vertebrate. Suitable vertebrates include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human.

### Kits, Systems, and Platforms

In another aspect of the current disclosure, kits, systems, and platforms are provided. In some embodiments, the kits systems and platforms comprise a polypeptide comprising from N to C terminus: an scFv specific for CD40 linked to IL-21; and a TLR9 agonist. The TLR agonist may be, for example, CpG. The kits, systems and platforms may further comprise reagents for isolating B cells from a sample, for example, reagents for negative selection of B cells from a blood leukapheresis sample.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

While some claims provided herein are directed to methods of treating a subject, both human and non-human subjects are envisioned. In addition, use of the compositions provided herein as medicaments for uses in therapy or for treating disease are also provided herein. Use of the compositions provided herein in the manufacture of a medicament for the treatment of a disease or condition are also encompassed.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

In the following example, the inventors describe the design of a fusion protein or polypeptide comprising CD40 scFV IL-21 (CD40ScFv-IL-21).

### Results

### Expression of CD40ScFv-IL-21

HEK293T cells (American Type Culture Collection, ATCC) were transiently transfected with fusion cDNA lentiviral plasmid using PolyFect (Qiagen). The supernatant was collected after 48 hours and concentrated with Amicon Centrifugal filter (Millipore). CD40ScFv-I-L21 expression level were quantified by human IL-21 ELISA (Biolegend).

### Biological Activity of Recombinant CD40ScFv-IL-21

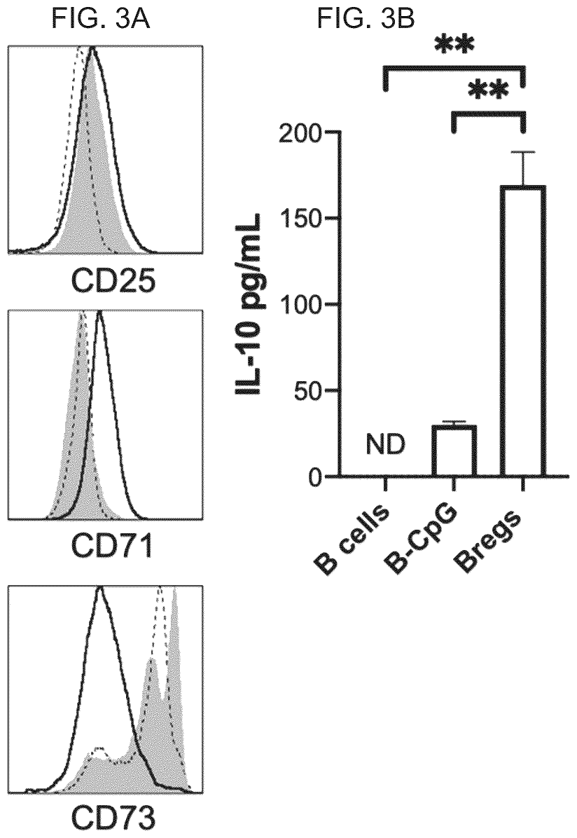

Biological activity was tested based on the ability of the recombinant fusion protein (e.g., CD40ScFv-IL-21) to convert B cells into IL-10 producing regulatory B cells (B_{regs}). Human B cells were isolated from peripheral blood mononuclear cells (PBMC) with EasySep human B cells Enrichment kit (StemCell technologies). Purified B cells were cultured in complete RPMI-1640 medium at 1 million cells per mL in the presence of fusion protein 100 ng/ml and 0.1 μM CpG (ODN2006, Invivogen) or CpG only. After 3-6 days cells were phenotypically characterized by flow cytometry for the expression of CD25, CD71 and CD73 on a Attune NxT Flow Cytometer (Thermofisher) (FIG. 3A).

IL-10 secreted was measured by IL-10 Elisa (eBioscience) (FIG. 3B). It is known that IL-10 is a key cytokine in modulating the immune response, and opposes the switch to the metabolic program induced by inflammatory stimuli in macrophages (15).

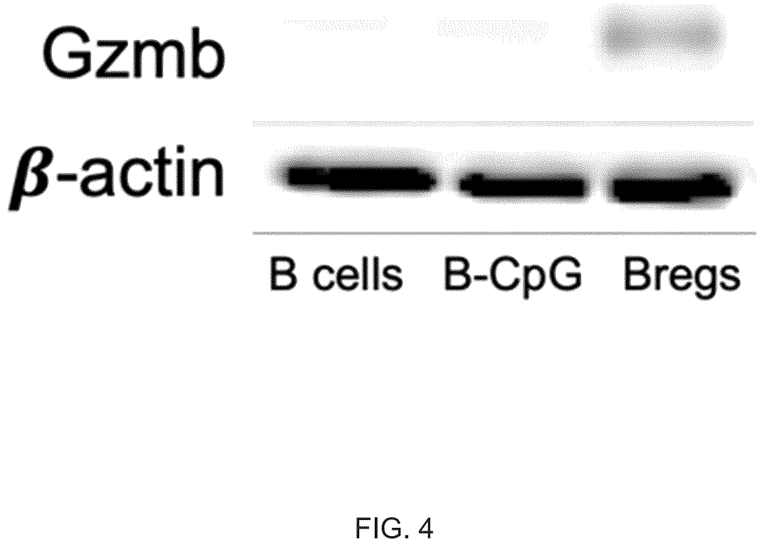

Expression of Granzyme B (Gzmb) was detected by Western blotting (FIG. 4). It is widely accepted in the field that B cell-derived Gzmb exert non-cytotoxic immunoregulatory effect by proteolytic activity on component expressed on the surface of target cells (e.g., T cell receptor (TCR)) (16).

### Monocyte Polarization

Human monocytes were isolated from human peripheral blood mononuclear cells (PBMCs) by negative selection (Thermofisher scientific). Isolated monocytes were cultured in T25 flasks at 0.5 million cells/mL per flask for 4 days. At day 4 B_{regs} or B-cpg cells were added to the monocytes culture at 1:1 ratio for 48 hours. At day 6 cells were collected and stained for CD14, CD16, CD163 and CD274 expression and analyzed by flow cytometer.

We found that B_{regs} can affect the phenotype of human blood-born macrophages by upregulation the expression of CD16 and CD163 (FIG. 5).

### Therapeutic Effect of B_{regs} in Radiation Induced-Toxicity

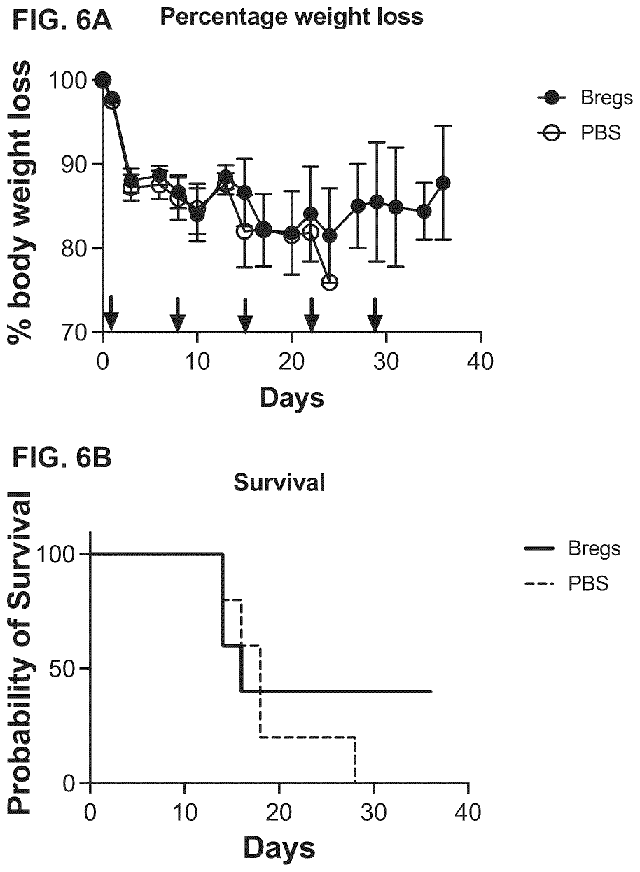

NSG mice were sublethally X-ray irradiated (3 Gy) 24 hours before the first intravenous (iv) injection of B_{regs} or PBS. Thereafter, mice received once a week iv injection of B_{regs} or PBS. The amount of B_{regs} injected per mouse was between 3 to 6 million in 200 μL of PBS. Body weight and survival was monitored over time (FIG. 6). Mice that received iv injection of B_{regs} showed overall better survival than the mice that received PBS.

### REFERENCES

1. Cerqueira, C., Manfroi, B., and Fillatreau, S. (2019) IL-10-producing regulatory B cells and plasmocytes: Molecular mechanisms and disease relevance. Semin Immunol 44, 101323
2. Pylayeva-Gupta, Y., Das, S., Handler, J. S., Hajdu, C. H., Coffre, M., Koralov, S. B., and Bar-Sagi, D. (2016) IL35-Producing B Cells Promote the Development of Pancreatic Neoplasia. Cancer Discovery 6, 247-255
3. Wang, R. X., Yu, C. R., Dambuza, I. M., Mahdi, R. M., Dolinska, M. B., Sergeev, Y. V., Wingfield, P. T., Kim, S. H., and Egwuagu, C. E. (2014) Interleukin-35 induces regulatory B cells that suppress autoimmune disease. Nature Medicine 20, 633-641
4. Blair, P. A., Norena, L. Y., Flores-Borja, F., Rawlings, D. J., Isenberg, D. A., Ehrenstein, M. R., and Mauri, C. (2010) CD19(+)CD24(hi)CD38(hi) B Cells Exhibit Regulatory Capacity in Healthy Individuals but Are Functionally Impaired in Systemic Lupus Erythematosus Patients. Immunity 32, 129-140
5. Yanaba, K., Bouaziz, J. D., Haas, K. M., Poe, J. C., Fujimoto, M., and Tedder, T. F. (2008) A regulatory B cell subset with a unique CD1d(hi)CD5(+) phenotype controls T cell-dependent inflammatory responses. Immunity 28, 639-650
6. Rosser, E. C., and Mauri, C. (2015) Regulatory B cells: origin, phenotype, and function. Immunity 42, 607-612
7. Habib, J., Deng, J., Lava, N., Tyor, W., and Galipeau, J. (2015) Blood B Cell and Regulatory Subset Content in Multiple Sclerosis Patients. J Mult Scler (Foster City) 2
8. Mauri, C., and Menon, M. (2017) Human regulatory B cells in health and disease: therapeutic potential. The Journal of Clinical Investigation 127, 772-779
9. Rafei, M., Wu, J. H., Annabi, B., Lejeune, L., Francois, M., and Galipeau, J. (2007) A GMCSF and IL-15 fusokine leads to paradoxical immunosuppression in vivo via asymmetrical JAK/STAT signaling through the IL-15 receptor complex. Blood 109, 2234-2242
10. Rafei, M., Hsieh, J., Zehntner, S., Li, M., Forner, K., Birman, E., Boivin, M. N., Young, Y. K., Perreault, C., and Galipeau, J. (2009) A granulocyte-macrophage colony-stimulating factor and interleukin-15 fusokine induces a regulatory B cell population with immune suppressive properties. Nat Med 15, 1038-1045
11. Pennati, A., Deng, J., and Galipeau, J. (2014) Maltose-binding protein fusion allows for high level bacterial expression and purification of bioactive mammalian cytokine derivatives. PLOS One 9, e106724
12. Pennati, A., Ng, S., Wu, Y., Murphy, J. R., Deng, J., Rangaraju, S., Asress, S., Blanchfield, J. L., Evavold, B., and Galipeau, J. (2016) Regulatory B Cells Induce Formation of IL-10-Expressing T Cells in Mice with Autoimmune Neuroinflammation. J Neurosci 36, 12598-12610
13. Pennati, A., Asress, S., Glass, J. D., and Galipeau, J. (2018) Adoptive transfer of IL-10(+) regulatory B cells decreases myeloid-derived macrophages in the central nervous system in a transgenic amyotrophic lateral sclerosis model. Cell Mol Immunol 15, 727-730
14. Pennati, A., Nylen, E. A., Duncan, I. D., and Galipeau, J. (2020) Regulatory B Cells Normalize CNS Myeloid Cell Content in a Mouse Model of Multiple Sclerosis and Promote Oligodendrogenesis and Remyelination. J Neurosci 40, 5105-5115
15. Ip, W. K. E., Hoshi, N., Shouval, D. S., Snapper, S., and Medzhitov, R. (2017) Anti-inflammatory effect of IL-10 mediated by metabolic reprogramming of macrophages. Science 356, 513
16. Hagn, M., and Jahrsdörfer, B. (2012) Why do human B cells secrete granzyme B? Insights into a novel B-cell differentiation pathway. Oncoimmunology 1, 1368-1375

### Example 2

In vitro T cell suppression assays are used to determine the suppressive capacity of cells or compounds by measuring their abilities to suppress the proliferation of responding T cells. The responder cells are labeled with a proliferation tracking antibody, Ki-67. The responder cells are incubated with the cells or compounds and stimulated for proliferation in the presence of immunomodulatory compounds. The proliferation of the responder cells in each treatment condition is tracked by flow cytometry after 3-4 days of activation.

In this assay, the inventors tested the suppression of phytohemagglutinin (PHA)-activated T cells with B cells, B_CpG or B_{regs} cells or conditioned media. The inventors isolated peripheral blood mononuclear cells (PBMCs) from whole human blood.

B_{regs} were generated by contacting human B cells with the disclosed recombinant fusion proteins, e.g., CD40ScFv-IL-21 as above in Example 1, and as described in the description above.

For Human T cell proliferation, T cells were labeled post-treatment with Ki-67 (also known as MKI67) a cellular marker for proliferation. Ki-67 is strictly associated with cell proliferation. During interphase, the Ki-67 antigen can be exclusively detected within the cell nucleus, whereas in mitosis most of the protein is relocated to the surface of the chromosomes.

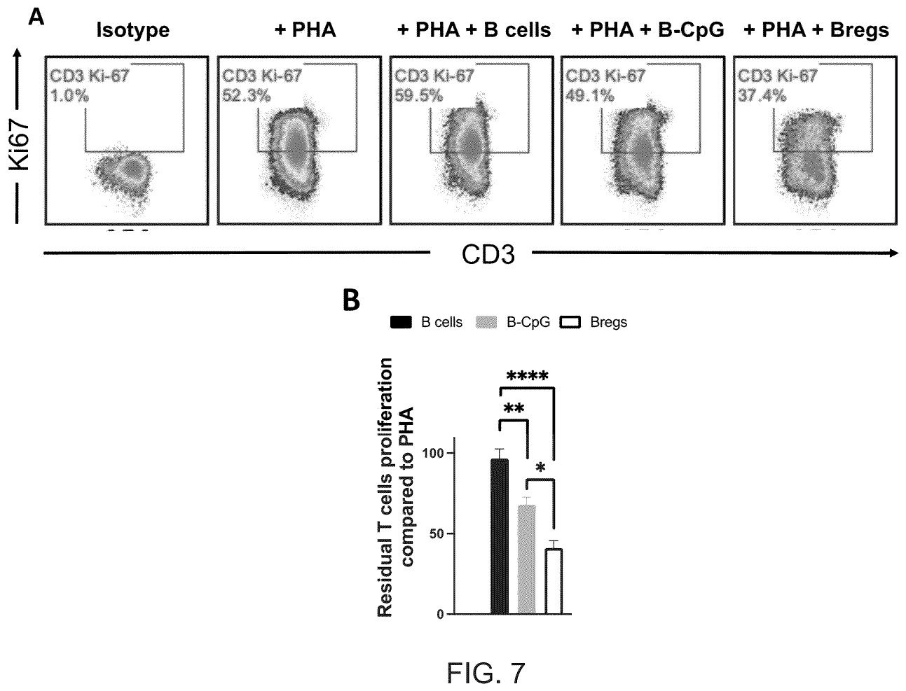

T cells were cultured in the presence of unstimulated B cells, B-CpG or B_{regs} in presence or absence of PHA. After 3 or 4 days the cells were collected, stained with a viable dye followed by surface staining with T cells markers. Proliferation was detected by flow cytometry (FIG. 7A). B_{regs} cells were significantly stronger to suppress activated T cells respect to unstimulated B cells or B-CpG (FIG. 7B). Thus, the disclosed recombinant fusion proteins were used to generate functional B_{regs} that suppressed T cell proliferation.

### Example 3

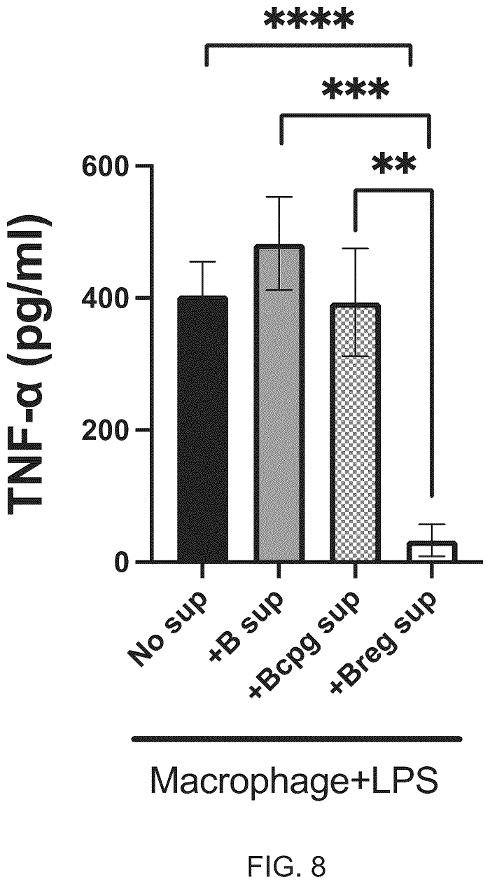

Depending on the microenvironment, macrophages can polarize to M1 (inflammatory) or M2 (anti-inflammatory) phenotypes. Human peripheral blood mononuclear cells (PBMCs) were separated by density centrifugation using Ficoll. CD19 and CD14 beads were used to negatively and positively select B cells and monocytes, respectively, according to the manufacturer's protocol. The purity of the cells was checked by flow cytometry after each isolation, and the typical purity for both B cells and monocytes is >98%. Monocytes-derived macrophages (MDM) were differentiated with 20 ng/ml of M-CSF for 3 days and replenished for another 2 days. Unstimulated B cells, B-CpG or B_{regs} conditioned media were added to the macrophages for 24 h. LPS was added to a concentration of 100 ng/ml for 24 after washing the cells with PBS. MDM conditioned media was tested for the expression of TNF-α. MDM treated with B_{regs} conditioned media (B_{reg} sup) showed the highest decreased in TNF-α expression compared to MDM treated with conditioned media from unstimulated cells (B sup) or B-CpG condition media (B-CpG sup) (FIG. 8). There is no statistical difference between MDM treated with conditioned media from unstimulated cells and conditioned media from B-CpG stimulated ones (FIG. 8).

Therefore, the disclosed recombinant fusion proteins were used to generate functional Bree that secreted soluble factors that altered macrophage polarization away from an M1-like phenotype.

What is claimed:

1. A polypeptide comprising from N to C terminus: an scFv specific for CD40 linked to IL-21.

2. The polypeptide of claim 1, wherein the scFv comprises SEQ ID NO: 1-6 or has 95% identity to SEQ ID NO: 1-6.

3. The polypeptide of any of claim 1 or 2, wherein the scFv comprises SEQ ID NO: 7 or has 90% identity to SEQ ID NO: 7.

4. The polypeptide of any one of the preceding claims, wherein the scFv is linked via a peptide bond to IL-21.

5. The polypeptide of any one of the preceding claims, wherein the scFv is linked to IL-21 via a linker peptide.

6. The polypeptide of claim 5, wherein the linker comprises SEQ ID NO: 8 or has 90% identity with SEQ ID NO: 8.

7. The polypeptide of any one of the preceding claims, wherein the polypeptide further comprises a signal peptide.

8. The polypeptide of claim 7, wherein the signal peptide comprises SEQ ID NO: 9 or has 90% identity with SEQ ID NO: 9.

9. The polypeptide of any one of the preceding claims, wherein IL-21 comprises SEQ ID NO: 10 or has 90% identity with SEQ ID NO: 10.

10. The polypeptide of any one of the preceding claims, wherein the polypeptide comprises SEQ ID NO: 11 or has 90% identity with SEQ ID NO: 11.

11. A pharmaceutical composition comprising the polypeptide of any one of claims 1-10 and a pharmaceutically acceptable carrier.

12. A method of generating B regulatory cells (B_{reg}) cells comprising contacting B cells isolated from a biological sample from a subject with the polypeptide of any one of claims 1-10 or the pharmaceutical composition of claim 11 and a TLR9 agonist for at least about 2 days in vitro.

13. The method of claim 12, wherein the TLR9 agonist is CpG.

14. The method of claim 12 or 13, wherein the cells are contacted for about 3-5 days.

15. The method of any one of claims 12-14, wherein the B cells are contacted with the polypeptide or the pharmaceutical composition and the TLR agonist at substantially the same time.

16. The method of any one of claims 12-15, wherein the B cells are contacted with the TLR agonist before the polypeptide or the pharmaceutical composition.

17. The method of any one of claims 12-16, wherein the biological sample is peripheral blood, plasma, or derived from leukapheresis.

18. The method of any one of claims 12-17, wherein the B cells are isolated from peripheral blood mononuclear cells (PBMCs) or from leukapheresis.

19. The method of any one of claims 12-18, wherein the B cells are isolated from a sample that has been cryopreserved.

20. The method of any one of claims 12-19, wherein the B cells are further cryopreserved after the contacting step.

21. A method of treating a subject, the method comprising administering cells generated by the method of any one of claims 12-20 to a subject in an amount sufficient to treat the subject, wherein the subject is in need of treatment with regulatory B cells.

22. The method of claim 21, wherein the subject is in need of treatment for an inflammatory or autoimmune disease or disorder, or a tissue injury syndrome.

23. The method of claim 22, wherein the inflammatory or autoimmune disease or disorder is selected from the group consisting of: multiple sclerosis (MS), inflammatory colitis, radiation injury, and amyotrophic lateral sclerosis (ALS).

24. The method of claim 23, wherein the subject is in need of treatment for MS, and wherein the method induces re-myelination of the peripheral nerves damaged by MS.

25. The method of any of claims 21-24, wherein the number of cells administered to the subject is about 1×10⁶ to 1×10⁷ cells/kg.

26. A construct comprising a polynucleotide encoding the polypeptide of any one of claims 1-10.

27. The construct of claim 26, further comprising a promoter operably linked to the polynucleotide.

28. The construct of claim 26 or 27, wherein the polynucleotide comprises SEQ ID NO: 13.

29. A cell comprising the construct of any one of claims 26-28 and capable of producing the polypeptide.

30. A kit, system, or platform comprising a polypeptide comprising from N to C terminus: an scFv specific for CD40 linked to IL-21, and a TLR9 agonist.

31. The kit, system, or platform of claim 30, wherein the TLR9 agonist comprises CpG.

32. A method of treating a subject comprising administering the polypeptide of any one of claims 1-10 or the composition of claim 11 to a subject in an amount sufficient to treat the subject, wherein the subject is in need of treatment with regulatory B cells.

33. The method of claim 32, wherein the subject is in need of treatment for an inflammatory or autoimmune disease or disorder, or a tissue injury syndrome.

34. The method of claim 33, wherein the inflammatory or autoimmune disease or disorder is selected from the group consisting of: multiple sclerosis (MS), inflammatory colitis, radiation injury, and amyotrophic lateral sclerosis (ALS).

35. The method of claim 34, wherein the subject is in need of treatment for MS, and wherein the method induces re-myelination of the peripheral nerves damaged by MS.
